(19) United States
(12) Patent Application Publication
(10) Pub. No.: US 2012/0296122 A1
(43) Pub. Date: Nov. 22, 2012

(54) FLUOROETHER-FUNCTIONALIZED AMINOAROMATIC COMPOUNDS AND DERIVATIVES THEREOF

(75) Inventors: Neville Everton Drysdale, Newark, DE (US); Fredrik Nederberg, Greenville, DE (US)

(73) Assignee: E. I. DU PONT DE NEMOURS AND COMPANY, Wilmington, DE (US)

(21) Appl. No.: 13/109,480

(22) Filed: May 17, 2011

(51) Int. Cl. C07C 209/36; C07C 211/52; C07C 205/37

(52) U.S. Cl. 564/417; 564/441

(57) ABSTRACT

Disclosed are fluoroether-functionalized aminoaromatic compounds and derivatives thereof. The compounds disclosed have utility as functionalized monomers and co-monomers in polyamides, polyoxadiazoles. Incorporation of the monomers into polymers can provide improved soil resistance to articles produced from the polymers.

### FIELD OF THE INVENTION

The present invention is directed to fluoroether-functionalized aminoaromatic compounds and derivatives thereof. The compounds disclosed have utility as functionalized monomers and co-monomers in, for example, polyamides and polyoxadiazoles.

### BACKGROUND

Fluorinated materials have many uses. In particular, they are used in polymer-related industries, and more particularly, in fiber-related industries, to impart soil, water and oil resistance, and improve flame retardancy. Generally, these materials are applied as topical treatments, but their effectiveness decreases over time due to material loss via wash and wear.

Thus, there is a need to provide polymeric materials with improved soil and oil resistance.

### SUMMARY OF THE INVENTION

In one aspect, the present invention provides a composition comprising a fluoroether-functionalized aminoaromatic compound represented by the structure (I)

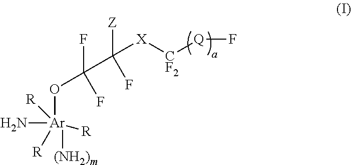

wherein,
Ar represents either a benzene or naphthalene radical;
m=0 or 1;
each R is independently H, C₁-C₁₀ alkyl, C₅-C₁₅ aryl, C₆-C₂₀ arylalkyl; OH, or a radical represented by the structure (II)

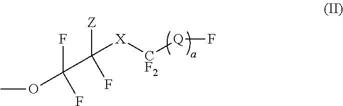

with the proviso that only one R can be OH or the radical represented by the structure (II);

### X is O or CF₂;

### Z is H, Cl, or Br;

Q represents the structure (Ia)

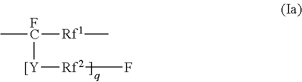

wherein
a=0 or 1;
q=0-10;
Y is O or CF₂;
Rf¹ is (CF₂)ₙ, wherein n is 0-10;
and,
Rf² is (CF₂)ₚ, wherein p is 0-10, with the proviso that when p is 0, Y is CF₂.

In another aspect, the present invention provides a process for preparing a fluoroether-functionalized aminoaromatic compound combining, a fluoroether-functionalized nitroaromatic compound of structure (VI)

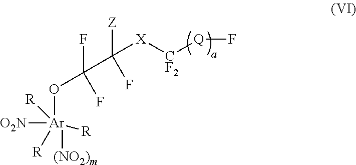

wherein,
Ar represents either a benzene or naphthalene radical;
m=0 or 1;
each R is independently H, C₁-C₁₀ alkyl, C₅-C₁₅ aryl, C₆-C₂₀ arylalkyl; OH, or a radical represented by the structure (II)

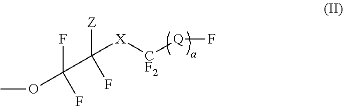

with the proviso that only one R can be OH or the radical represented by the structure (II);

### X is O or CF₂;

### Z is H, Cl, or Br;

Q represents the structure (Ia)

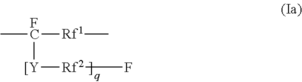

wherein
a=0 or 1;
q=0-10;
Y is O or CF₂;
Rf¹ is (CF₂)ₙ, wherein n is 0-10;
and,
Rf² is (CF₂)ₚ, wherein p is 0-10, with the proviso that when p is 0, Y is CF₂;
   in a sealable vessel and under hydrogen pressure, with one or more solvents and a catalyst to form a reaction mixture, stirring the reaction mixture at room temperature for a period of time sufficient to form a fluoroether-functionalized aminoaromatic compound of structure (I), wherein Ar, m, R, X, Z, Q, q, Y, Rf¹, Rf², n, and p are as stated above.

### DETAILED DESCRIPTION

When a range of numerical values is provided herein, it is intended to encompass the end-points of the range unless specifically stated otherwise. Numerical values used herein have the precision of the number of significant figures provided, following the standard protocol in chemistry for significant figures as outlined in ASTM E29-08 Section 6. For example, the number 40 encompasses a range from 35.0 to 44.9, whereas the number 40.0 encompasses a range from 39.50 to 40.49.

As used herein, the term “fluoroether-functionalized aromatic compound” refers to the compounds of structures (I and VI). The term “fluoroether-functionalized aminoaromatic compound” refers to that subclass of compounds of structure (I). The term “fluoroether-functionalized nitroaromatic compound” refers to that subclass of compounds of structure (VI).

In one aspect, the present invention provides a composition comprising a fluoroether-functionalized aminoaromatic compound represented by the structure (I)

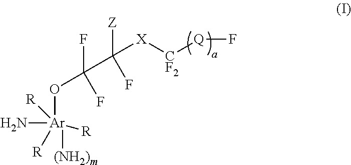

wherein,
Ar represents a benzene or naphthalene radical;
m=0 or 1;
each R is independently H, C₁-C₁₀ alkyl, C₅-C₁₅ aryl, C₆-C₂₀ arylalkyl; OH, or a radical represented by the structure (II)

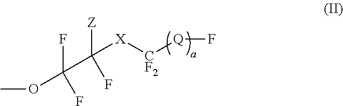

with the proviso that only one R can be OH or the radical represented by the structure (II);

### X is O or CF₂;

### Z is H, Cl, or Br;

Q represents structure (Ia)

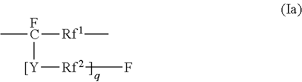

wherein
a=0 or 1;
q=0-10;
Y is O or CF₂;
Rf¹ is (CF₂)ₙ, wherein n is 0-10;
and,
Rf² is (CF₂)ₚ, wherein p is 0-10, with the proviso that when p is 0, Y is CF₂.

In one embodiment, the compound is represented by the structure (IVa).

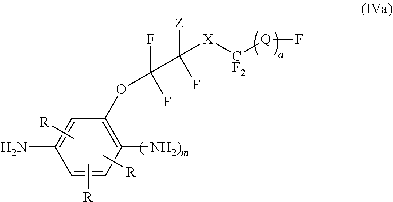

wherein R, Z, X, Q, m and a are as recited supra.

In another embodiment, the compound is represented by the structure (IVb).

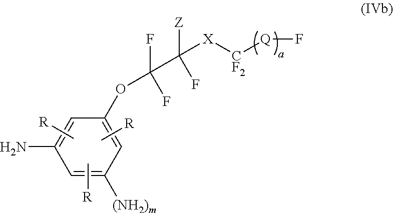

wherein R, Z, X, Q, m and a are as recited supra.

As can be noted in the structures above, the substituents can be attached to the aromatic ring at any point, thus making it possible to have ortho-, meta- and para-substituents as defined above.

In one embodiment, m=1;

In one embodiment, one R is OH.

In one embodiment, each R is H.

In one embodiment, one R is OH and the remaining two Rs are each H.

In one embodiment, one R is represented by the structure (II) and the remaining two Rs are each H.

In one embodiment, X is O. In an alternative embodiment, X is CF₂.

In one embodiment, Y is O. In an alternative embodiment, Y is CF₂.

In one embodiment Z is Cl or Br. In a further embodiment, Z is Cl. In an alternative embodiment, one R is represented by the structure (II), and one Z is H. In a further embodiment, one R is represented by the structure (II), one Z is H, and one Z is Cl.

In one embodiment, Rf¹ is CF₂.

In one embodiment, Rf² is CF₂.

In one embodiment, Rf² is a bond (that is, p=0), and Y is CF₂.

In one embodiment, each R is H, Z is Cl, X is O, Y is O, Rf¹ is CF₂, and Rf² is perfluoropropenyl, and q=1.

In one embodiment, a=0.

In one embodiment, a=1, q=0, m=1, and n=0.

In one aspect, the present invention provides a process for preparing the fluoroether-functionalized aminoaromatic compounds.

In one embodiment, the reaction of reducing the fluoroether-functionalized nitroaromatic compound of the structure (VI) to the fluoroether-functionalized aminoaromatic compound of the structure (I) is performed while agitating the reaction mixture. In one embodiment, the reaction occurs at a temperature above room temperature but below the reflux temperature of the reaction mixture, and the reaction mixture is cooled following reaction. The reaction mixture can be held at the reaction temperature until the desired yield of reaction is achieved.

Suitable catalysts for the reduction of fluoroether-functionalized nitroaromatic to fluoroether-functionalized aminoaromatic compound include, Palladium, Platimium, Iron, nickel sulfide, a catalyst that consist of Cu, Cr, Ba, and Zinc oxide or other catalyst systems known to one skilled in the art. The reduction of the fluoroether-functionalized nitroaromatic compound to the fluoroether-functionalized aminoaromatic compound can be terminated by cooling and releasing the hydrogen pressure.

In the practice of the process for preparation of fluoroether-functionalized aminoaromatic compound, the fluoroether-functionalized nitroaromatic compound, is contacted with hydrogen, under pressure, in the presence of a catalyst and a solvent at room temperature for a length of time sufficient to provide the desired quantity of product. The length of time can be from a few minutes to several hours depending on catalyst and catalyst concentration, and the desired yield. In one embodiment the process further comprises contacting the fluoroether-functionalized nitroaromatic compound with one or more solvents in the presence of a suitable catalyst. Suitable solvents include methanol, ethanol, water, terahydrofuran and other solvents known in the art. Reduction can be performed at concentrations ranging from 0.5 to 5.00 M concentration of the nitroaromatic compound in the solvent. The catalyst concentration can vary from 0.5 to 10 weight percent of catalyst relative to the weight of the starting nitroaromatic compound. The hydrogen pressure suitable for the present process is from 12 to 10,000 psi. Preferably, the hydrogen pressure can be from 12 to 1,000 psi, more preferably, the hydrogen pressure can be from 12-500 psi.

In one embodiment, the reaction for preparation of fluoroether-functionalized aminoaromatic compound is continued until no further product is produced over some pre-selected time scale. The required reaction time to achieve the desired degree of conversion depends upon the reaction temperature, the chemical reactivity of the specific reaction mixture components, and the degree of mixing applied to the reaction mixture, and can be readily determined by one skilled in the art. Progress of the reaction can be monitored using any one of a variety of established analytical methods, including, but not limited to, nuclear magnetic resonance spectroscopy, thin layer chromatography (TLC), and gas chromatography (GC). When the desired level of conversion has been achieved, the reaction mixture is quenched, as described supra. In one embodiment, the thus quenched reaction mixture is filtered and the filtrate concentrated under reduced pressure. In one embodiment, a plurality of compounds encompassed by the structure (I) can be made in a single reaction mixture. In such cases, separation of the products thus produced can be effected by any method known to the skilled artisan such as, for example, distillation or column chromatography.

Suitable fluoroether-functionalized nitroaromatic compounds for the preparation of the fluoroether-functionalized aminoaromatic compounds are represented by the structure (VI).

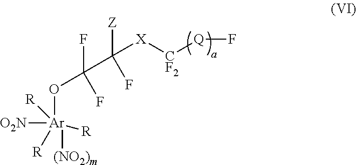

wherein,
Ar represents either a benzene or a naphthalene radical;
m=0 or 1;
each R is independently H, C₁-C₁₀ alkyl, C₅-C₁₅ aryl, C₆-C₂₀ arylalkyl; OH, or a radical represented by the structure (II)

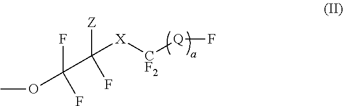

with the proviso that only one R can be OH or the radical represented by the structure (II);

### X is O or CF₂;

### Z is H, Cl, or Br;

Q represents structure (Ia)

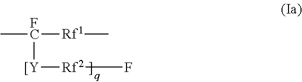

wherein
a=0 or 1;
q=0-10;
Y is O or CF₂;
Rf¹ is (CF₂)ₙ, wherein n is 0-10;
and,
Rf² is (CF₂)ₚ, wherein p is 0-10, with the proviso that when p is 0, Y is CF₂.

In one embodiment, the suitable fluoroether-functionalized nitroaromatic compound is represented by the structure (IVa).

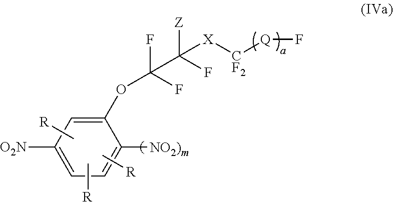

wherein R, Z, X, Q, m and a are as recited supra.

In another embodiment, the suitable fluoroether-functionalized nitroaromatic compound is represented by the structure (IVb).

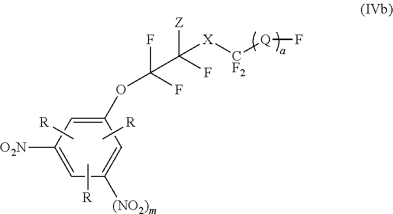

wherein R, Z, X, Q, m and a are as recited supra.

As can be noted in the structures above, the substituents can be attached to the aromatic ring at any point, thus making it possible to have ortho-, meta- and para-substituents as defined above.

In one embodiment, m=1;

In one embodiment, one R is OH.

In one embodiment, each R is H.

In one embodiment, one R is OH and the remaining two Rs are each H.

In one embodiment, one R is represented by the structure (II) and the remaining two Rs are each H.

In one embodiment, X is O. In an alternative embodiment, X is CF₂.

In one embodiment, Y is O. In an alternative embodiment, Y is CF₂.

In one embodiment Z is Cl or Br. In a further embodiment, Z is Cl.

In an alternative embodiment, one R is represented by the structure (II), and one Z is H. In a further embodiment, one R is represented by the structure (II), one Z is H, and one Z is Cl.

In one embodiment, Rf¹ is CF₂.

In one embodiment, Rf² is CF₂.

In one embodiment, Rf² is a bond (that is, p=0), and Y is CF₂.

In one embodiment, each R is H, Z is Cl, X is O, Y is O, Rf¹ is CF₂, and Rf² is perfluoropropenyl, and q=1.

In one embodiment, a=0.

In one embodiment, a=1, q=0, m=1, and n=0.

In another aspect, the suitable fluoroether-functionalized nitroaromatic compound can be prepared by a process comprising forming a reaction mixture by contacting a hydroxy nitroaromatic compound in the presence of a solvent and a catalyst with a perfluorovinyl compound represented by the structure (III)

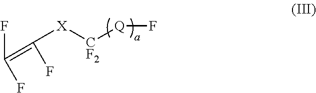

wherein X is O or CF₂, and Q represents the structure (Ia)

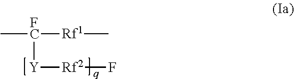

wherein
a=0 or 1;
q=0-10;
Y is O or CF₂;
Rf¹ is (CF₂)ₙ, wherein n is 0-10;
Rf² is (CF₂)ₚ, wherein p is 0-10, with the proviso that when p is 0, Y is CF₂;
   at a temperature within the range of about −70° C. to the reflux temperature of the reaction mixture. The fluoroether-functionalized nitroaromatic compound thus formed can be then reduced, by the process described above, to produce the desired fluoroether-functionalized aminoaromatic compound.

In one embodiment, the reaction mixture for forming the fluoroether-functionalized nitroaromatic compound is agitated during reaction. In one embodiment, the reaction occurs at a temperature above room temperature but below the reflux temperature of the reaction mixture, and the reaction mixture is cooled following reaction. The reaction mixture can be held at the reaction temperature until the desired yield of reaction is achieved.

In one embodiment, the solvent for forming the fluoroether-functionalized nitroaromatic compound is halogenated, and the process forms a fluoroether-functionalized nitroaromatic compound, in which Z is the corresponding halogen. Suitable halogenated solvents include but are not limited to methylene chloride, tetrachloromethane, tetrabromomethane, hexachloroethane and hexabromoethane. In an alternative embodiment, the solvent is non-halogenated, and in the resulting fluoroether-functionalized nitroaromatic compound, Z is H. Suitable non-halogenated solvents include but are not limited to tetrahydrofuran (THF), dioxane, and dimethylformamide (DMF). Thus, the reactions in the processes herein can be carried out in the presence of a chlorinating reagent that is volatile and can function as both a solvent and a chlorinating agent. Non-halogenated solvents are optional.

The reaction for forming the fluoroether-functionalized nitroaromatic compound is catalyzed by a base. A variety of basic catalysts can be used, i.e., any catalyst that is capable of deprotonating phenol. That is, a suitable catalyst is any catalyst having a pKa greater than that of phenol (9.95, using water at 25° C. as reference). Suitable catalysts include, but are not limited to, sodium methoxide, calcium hydride, sodium metal, potassium methoxide, potassium t-butoxide, potassium carbonate, benzyltrimethylammonium hydroxide, and sodium carbonate. Preferred are potassium t-butoxide, potassium carbonate, sodium carbonate and benzyltrimethylammonium hydroxide.

The reaction for forming the fluoroether-functionalized nitroaromatic compound can be terminated at any desirable point by the addition of acid (such as, for example, 10% HCl). Alternatively, when using solid catalysts, such as the carbonate catalysts, the reaction mixture can be filtered to remove the catalyst, thereby terminating the reaction.

Suitable hydroxy nitroaromatic compounds for forming the fluoroether-functionalized nitroaromatic compound include but are not limited to mononitrophenols, mononitro diphenols, dinitrodiphenols. Suitable mononitrophenols include but are not limited to 2-nitrophenol, 3-nitrophenol, or 4-nitrophenol. Suitable mononitro diphenols include but are not limited to 2-nitrobenzene-1,4-diol, 3-nitrobenzene-1,4-diol, 4-nitrobenzene-1,3-diol, 5-nitrobenzene-1,3-diol, 3-nitrobenzene-1,2-diol, 2-nitrobenzene-1,3-diol, 2,5-dinitrophenol, 3,5-dinitrophenol, 2,3-dinitrophenol, 3,4-dinitrophenol, 2,6-dinitrophenol. Suitable dinitrodiphenols include but are not limited to 2,5-dinitrobenzene-1,4-diol, 2,5-dinitrobenzene-1,3-diol, 3,6-dinitrobenzene-1,2-diol, 2,6-dinitrobenzene-1,4-diol, 3,5-dinitrobenzene-1,2-diol, 2,3-dinitrobenzene-1,4-diol, 4,5-dinitrobenzene-1,3-diol, 3,4-dinitrobenzene-1,2-diol, 4,5-dinitrobenzene-1,2-diol,

Suitable perfluorovinyl compounds for forming the fluoroether-functionalized nitroaromatic compound include, but are not limited to, 1,1,1,2,2,3,3-heptafluoro-3-(1,1,1,2,3,3-hexafluoro-3-(1,2,2-trifluorovinyl-ox)propan-2-yloxy)propane, heptafluoropropyltrifluorovinyl-ether, perfluoropent-1-ene, perfluorohex-1-ene, perfluorohept-1-ene, perfluorooct-1-ene, perfluoronon-1-ene, perfluorodec-1-ene, and mixtures thereof. In one embodiment, the perfluorovinyl compound is 1,1,1,2,2,3,3-heptafluoro-3-(1,1,1,2,3,3-hexafluoro-3-(1,2,2-trifluorovinyl-oxy)propan-2-yloxy)propane. In an alternative embodiment the perfluorovinyl compound is heptafluoropropyl-trifluorovinylether.

In the practice of the process for forming the fluoroether-functionalized nitroaromatic compound, a suitable hydroxy nitroaromatic compound and a suitable perfluorovinyl compound are contacted in the presence of a suitable solvent and a suitable catalyst until the reaction has achieved the desired degree of conversion. In one embodiment, the reaction is continued until no further product is produced over some pre-selected time scale. The required reaction time to achieve the desired degree of conversion depends upon the reaction temperature, the chemical reactivity of the specific reaction mixture components, and the degree of mixing applied to the reaction mixture, and can be readily determined by one skilled in the art. Progress of the reaction can be monitored using any one of a variety of established analytical methods, including, but not limited to, nuclear magnetic resonance spectroscopy, thin layer chromatography, and gas chromatography. When the desired level of conversion has been achieved, the reaction mixture is quenched, as described supra. In one embodiment, the thus quenched reaction mixture is concentrated under vacuum, and rinsed with a solvent. In one embodiment, a plurality of compounds encompassed by the structure (I) can be made in a single reaction mixture. In such cases, separation of the products thus produced can be effected by any method known to the skilled artisan such as, for example, distillation or column chromatography.

In one embodiment the process for forming the fluoroether-functionalized nitroaromatic compound further comprises contacting the hydroxy nitroaromatic compound with one or more solvents in the presence of a suitable catalyst. The suitable perfluorovinyl compound is then added to the solution and the reaction is allowed to proceed at room temperature for a period of time.

Once the fluoroether-functionalized aminoaromatic compound has been prepared, it is suitable for polymerization and other potential uses such as intermediates for surface protection compositions, pharmaceutical and agricultural chemicals. For example, this material is useful for making polymers such as aramids. An aramid polymer can be prepared by contacting a fluoroether-functionalized di-aminoaromatic compound with an equivalent amount of a diacid chloride such terephthaloyl chloride under nitrogen in an amide solvent such as dimethyl acetamide. The resulting polymer can be isolated by precipitation in water. The fluoroether-functionalized aminoaromatic compounds can also be used in combination with aliphatic diamines, such as hexamethylene diamine, to give new polyamide compositions. Thus various amounts, in moles, of the aliphatic diamine can be substituted with the fluoroether-functionalized aminoaromatic compound and then reacted with an aliphatic di-acid such as adipic acid or adipate ester, after polymerization at a elevated temperature, the resulting polymer can be isolated after cooling.

The invention is further described and illustrated in, but not limited to, the following specific embodiments.

### EXAMPLES

The chemicals and reagents were used as received in the Examples as follows:

Benzyltrimethylammonium hydroxide was obtained from Sigma-Aldrich.

1,1,1,2,2,3,3,heptafluorp-3-(1,1,1,2,3,3-hexafluoro-3-(1,2,2-trifluoro-vinyloxy)propane was obtained from SynQuest Labs, Alachua, Fla.

### Example 1

### Preparation of 1,4-dinitro-2-(1,1,2-trifluoro-2-(1,1,2,3,3,3-hexafluoro-2(perfluoropropoxy)propoxy)ethoxybenzene

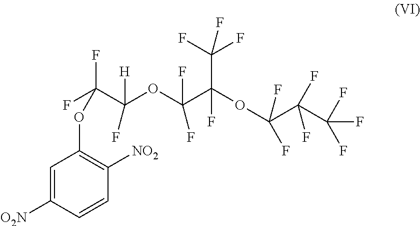

In a dry box, THF (25 mL), methylene chloride (25 mL) and 2,5-dinitrophenol (80%) (1.15 g, 0.005 mol) were added to an oven dry roundbottom flask equipped with a stirrer and benzyltrimethylammonium hydroxide (0.575, 0.0014 mol) was added. 1,1,1,2,2,3,3-Heptafluoro-3-(1,1,1,2,3,3-hexafluoro-3-(1,2,2-trifluorovinyloxy)propan-2-yloxy)propane, (SynQuest Labs, Alachua, Fla.), (25.40 g g, 0.0125 mol) was then added via an addition funnel and the reaction allowed to stir at room temperature. After 4 days the reaction was terminated via addition of 1.0 mL of 10% HCl, concentrated under reduced pressure and was purified using column chromatography to give 1.02 g (33.17% yield) of the desired material, 1,4-dinitro-2-(1,1,2-trifluoro-2-(1,1,2,3,3,3-hexafluoro-2-(perfluoropropoxy)propoxy ethoxy)benzene (structure VI).

### Example 2

### Preparation of 2-(1,1,2-trifluoro-2-(1,1,2,3,3,3-hexafluoro-2-(perfluoropropoxy)propoxy)ethoxy)benzene-1,4-diamine

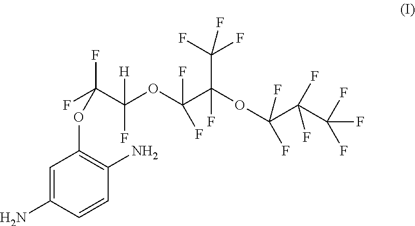

In a Fischer Porter tube (75 mL), was added Pt/C (0.25 g) followed by a solution of 1,4-dinitro-2-(1,1,2-trifluoro-2-(1,1,2,3,3,3-hexafluoro-2-(perfluoropropoxy)propoxy)ethoxy)benzene (0.70 g) (prepared as described in Example 1), methanol (10.0 mL) and water (2.5 mL). The tube was sealed and hydrogen was introduced to a pressure of 40 psi. The reaction was stirred at room temperature for six days. The catalyst was removed by filtration and the solution concentrated at reduced pressure and column chromatograph to obtain the desired material, 2-(1,1,2-trifluoro-2-(1,1,2,3,3,3-hexafluoro-2-(perfluoropropoxy)propoxy)ethoxy)benzene-1,4-diamine (structure I). Rf=0.11 (hexane (4)/THF(1), by volume).

What is claimed is:

1. A composition comprising a fluoroether-functionalized aminoaromatic compound represented by the structure (I) 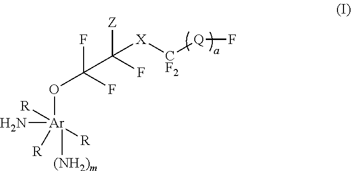
wherein,
Ar represents either a benzene or naphthalene radical;
m=0 or 1;
each R is independently H, C₁-C₁₀ alkyl, C₅-C₁₅ aryl, C₆-C₂₀ arylalkyl, OH, or a radical represented by the structure (II) 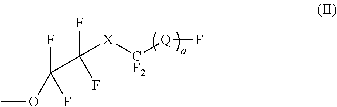
with the proviso that only one R can be OH or the radical represented by the structure (II);
X is O or CF₂;
Z is H, Cl, or Br;
Q represents the structure (Ia) 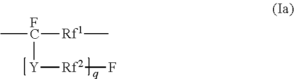
wherein
a=0 or 1;
q=0-10;
Y is O or CF₂;
Rf¹ is (CF₂)ₙ, wherein n is 0-10; and
Rf² is (CF₂)ₚ, wherein p is 0-10, with the proviso that when p is 0, Y is CF₂.

2. The composition of claim 1 wherein m=1.

3. The composition of claim 1 wherein Ar is a benzene radical.

4. The composition of claim 1 wherein each R is H.

5. The composition of claim 1 wherein one R is a radical represented by the structure (II).

6. The composition of claim 1 wherein at least one R is C₁-C₁₀ alkyl, C₅-C₁₅ aryl, or C₆-C₂₀ arylalkyl.

7. The composition of claim 1 wherein Z is Cl or Br.

8. The composition of claim 1 wherein each m=1, a=1, R is H, Z is Cl, X is O, Y is CF₂, n=1, p=0, and q=1.

9. The composition of claim 1 wherein a=0.

10. The composition of claim 1 wherein a=1, q=0, and n=0.

11. A process comprising combining a fluoroether-functionalized nitroaromatic compound of structure (VI) in the presence of a solvent and a catalyst 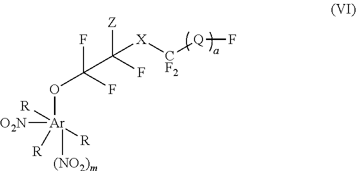
wherein,
Ar represents either a benzene or a naphthalene radical;
m=0 or 1;
each R is independently H, C₁-C₁₀ alkyl, C₅-C₁₅ aryl, C₆-C₂₀ arylalkyl, OH, or a radical represented by the structure (II) 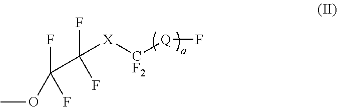
with the proviso that only one R can be OH or the radical represented by the structure (II);
X is O or CF₂;
Z is H, Cl, or Br;
Q represents the structure (Ia) 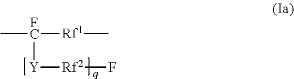
wherein
a=0 or 1;
q=0-10;
Y is O or CF₂;
Rf¹ is (CF₂)ₙ, wherein n is 0-10; and
Rf² is (CF₂)ₚ, wherein p is 0-10, with the proviso that when p is 0, Y is CF₂, in a sealable vessel and under hydrogen pressure, with one or more solvents and a catalyst to form a reaction mixture, stirring the reaction mixture at room temperature for a period of time sufficient to form a fluoroether-functionalized aminoaromatic compound of structure (I), 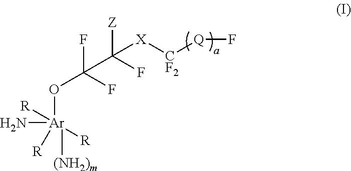
wherein,
Ar represents either a benzene or naphthalene radical;
m=0 or 1;
each R is independently H, C₁-C₁₀ alkyl, C₅-C₁₅ aryl, C₆-C₂₀ arylalkyl; OH, or a radical represented by the structure (II) 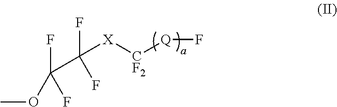
with the proviso that only one R can be OH or the radical represented by the structure (II);
X is O or CF₂;
Z is H, Cl, or Br;
Q represents the structure (Ia) 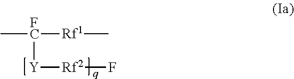
wherein
a=0 or 1;
q=0-10;
Y is O or CF₂;
Rf¹ is (CF₂)ₙ, wherein n is 0-10; and
Rf² is (CF₂)ₚ, wherein p is 0-10, with the proviso that when p is 0, Y is CF₂.

12. The process of claim 11 wherein the catalyst is palladium.

13. The process of claim 11 wherein the solvent is a mixture of methanol and water.

14. The process of claim 11 wherein the aromatic nitro portion of the fluoroether-functionalized nitroaromatic compound is selected from the group consisting of 2-nitrophenol, 3-nitrophenol, 4-nitrophenol, 2-nitrobenzene-1,4-diol, 3-nitrobenzene-1,4-diol, 4-nitrobenzene-1,3-diol, 5-nitrobenzene-1,3-diol, 3-nitrobenzene-1,2-diol, 2-nitrobenzene-1,3-diol, 2,5-dinitrophenol, 3,5-dinitrophenol, 2,3-dinitrophenol, 3,4-dinitrophenol, 2,6-dinitrophenol, 2,5-dinitrobenzene-1,4-diol, 2,5-dinitrobenzene-1,3-diol, 3,6-dinitrobenzene-1,2-diol, 2,6-dinitrobenzene-1,4-diol, 3,5-dinitrobenzene-1,2-diol, 2,3-dinitrobenzene-1,4-diol, 4,5-dinitrobenzene, 1,3-diol, 3,4-dinitrobenzene-1,2-diol, 4,5-dinitrobenzene-1,2-diol and mixtures thereof.

15. The process of claim 11 wherein the fluoroether-functionalized nitroaromatic compound is 1,4-dinitro-2-(1,1,2-trifluoro-2-(1,1,2,3,3,3-hexafluoro-2-(perfluoropropoxy)propoxy)ethoxy)benzene and wherein reduction of said compound produces 2-(1,1,2-trifluoro-2-(1,1,2,3,3,3-hexafluoro-2-(perfluoropropoxy)propoxy)ethoxy)benzene-1,4-diamine.
